(19) United States
(12) Patent Application Publication
(10) Pub. No.: US 2014/0051985 A1
(43) Pub. Date: Feb. 20, 2014

(54) PERCUTANEOUS NEPHROLITHOTOMY TARGET FINDING SYSTEM

(71) Applicants: Tailin Fan, Nashua, NH (US); Christopher A. Cook, New York, NY (US)

(72) Inventors: Tailin Fan, Nashua, NH (US); Christopher A. Cook, New York, NY (US)

(21) Appl. No.: 13/913,649

(22) Filed: Jun. 10, 2013

Related U.S. Application Data
(60) Provisional application No. 61/684,222, filed on Aug. 17, 2012.
(60) Provisional application No. 61/783,607, filed on Mar. 14, 2013.

(51) Int. Cl. A61B 1/00; A61B 17/34; A61B 1/307

(52) U.S. Cl. CPC A61B 1/00158; A61B 1/00004; A61B 1/307; A61B 17/3403
USPC 600/424

(57) ABSTRACT

A target finding system identifies a surgical target such as a kidney stone by disposing an emitter such as a magnetic source behind or adjacent the surgical target, and employing a circuit to identify an axis to the emitter, thus defining an axis or path to the surgical target. An array of sensors arranged in an equidistant, coplanar arrangement each senses a signal indicative of a distance to the emitter. A magneto resistor sensor generates a variable resistance is responsive to the distance to a magnetic coil emitting a magnetic field. An equal signal from each of the coplanar sensors indicates positioning on an axis passing through a point central to the sensors and orthogonal to the plane. In one embodiment, a pair of Wheatstone bridges enhances the accuracy of the system.

### RELATED APPLICATIONS

This application claims the benefit of the filing date of earlier filed pending U.S. Provisional Patent Application having Ser. No. 61/684,222, filed Aug. 17, 2012, and entitled “PERCUTANEOUS NEPHROLITHOTOMY TARGET FINDING SYSTEM,” and earlier filed pending U.S. Provisional Patent Application having Ser. No. 61/783,607, filed Mar. 14, 2013, and entitled “SURGICAL POSITIONING CIRCUIT.” The entire teachings and contents of these Provisional Patent Applications are hereby incorporated by reference herein in their entireties.

### BACKGROUND

Surgical procedures often rely on precise positioning to locate particular surgical targets without disturbing nearby structures that may adversely affect the surgical results. Open field surgery relies on a steady hand and sharp eye of a surgeon for ensuring that surgical intervention is focused accordingly. In recent decades, endoscopic and laparoscopic techniques perform minimally invasive procedures with elongated instruments by working through small incisions, rather than a large open field. Many procedures involve simply locating and removing a harmful growth or structure, such as a tumor, cyst or polyp. In the case of kidney stones, for example, the stone may be broken up and removed, once located with endoscopic equipment.

Various systems exist to detect and track instruments used in medical procedures. For example, ScopeGuide® manufactured by Olympus Medical is a visualization tool, used in gastroenterology to visualize the shape of the endoscope's shaft inside a patient's body. Many magnetic sources along the endoscope's shaft are used in conjunction with detectors which are coil arrays. The magnetic detecting elements are located in the operating table/taped on patient's skin/inside receiver dish. The system includes an extensively distributed, instrumented bed/skin/receiver dish, processing unit and requires multi-channel, digital processing.

ScopeGuide® employs magnetic endoscopic imaging (MEI) to produce a three-dimensional view of the scope and its location within the colon in real-time. A number of small electromagnetic transmission coils located within the colonoscope itself generate a weak magnetic field, which is picked up by the ScopeGuide receiver. The received signals allow the ScopeGuide processor to calculate the location and orientation of each transmission coil, which is used to generate the 3D rendering of the scope, displayed on a screen. A separate handheld 3D marker helps an assistant to visualize where abdominal pressure will be most effective.

Another Olympus technology, ScopeGuide's 3D visualization allows the endoscopist to see loop formations as they are occurring for quicker and more effective loop management. It can also assist with scope insertion and help optimize scope handling, which may shorten procedure times and minimize patient discomfort, even during difficult colonoscopies.

Other conventional systems for tracking instruments within the body are very complex and require extensive signal processing. Robert Marcovich & Arthur D. Smith, Percutaneous Renal Access: Tips and Tricks. BJU International, 2005, page 78, states: “The first requisite for percutaneous access is some sort of guidance system. The renal puncture can be made under ultrasonographic, fluoroscopic or CT control.”

Li-Ming Su, MD, et al. Robotic Percutaneous Access to the Kidney: Comparison with Standard Manual Access. J. Endourology 2002; 16:7, page 471 states: “The task of obtaining access manually requires substantial skill and can be particularly difficult when the collecting system is not dilated. Inaccurate placement of the needle can risk injuring the kidney and adjacent organs, thus compromising the planned percutaneous procedure, as well as the clinical outcome of the patient. For these reasons, coupled with the limitations in C-arm fluoroscopy in providing only a continuous two-dimensional view of the collecting system, obtaining renal access in the operating room remains a challenging task for many urologists. Many institutions have relinquished this task to the skills of interventional radiologists to perform under fluoroscopic, ultrasonographic, or CT guidance.” The cited references are incorporated by reference in their entireties. Robotic percutaneous renal access has been performed relying on fluoroscopy for guidance.

### SUMMARY

Embodiments disclosed herein facilitate the alignment of a percutaneous nephrolithotomy needle with a targeted calyx (see FIGS. 1 and 2). This is a very important step during percutaneous nephrostomy, which prepares for a percutaneous nephrolithotomy (PCNL) procedure. PCNL target finding TF system uses magnetism as the physical property to sense.

In one embodiment, a technique for finding a target includes providing a first magnetic source disposed at an end of an endoscope and having an electromagnetic signal in the vicinity of a target, detecting the electromagnetic signal using a sensor array having at least four sensors arranged in a bridge configuration, determining a position and orientation of a needle guide aperture relative to the target and indicating the position and orientation of the needle guide aperture relative to the electromagnetic signal in the vicinity of the target. Such a technique provides a much quicker and more accurate percutaneous renal access, which reduces the risk associated with radiation visualization, the time and cost spent on the procedure, patient blood loss due to multiple/faulty attempted access and the probability of complications due to multiple/faulty attempted access.

In a further embodiment, a target finding system, includes an internal target object (ITO) disposed at an end of an endoscope providing an electromagnetic signal and an external target finder (ETF). The ETF includes a needle guide aperture and at least four sensors arranged symmetrically around the needle guide aperture, the sensors configured to receive the electromagnetic signal from the ITO. The system further includes a detector coupled to the at least four sensors to determine an orientation and a position of a needle guide aperture relative to the ITO and a user interface connected to the ETF to indicate the orientation and the position of the needle guide aperture relative to the ITO. Such a system, while not focused on producing absolute 3D space position/orientation of a magnetic field source, is able to determine the relative orientation and alignment between a magnetic source and PCNL needle in a less computationally involved way. The implementation may be either with analog or digital means in a simple hand-held device, provided with features that make it user-friendly. Such a device may be manufactured and sold in a low-cost way and would therefore be more readily available to the medical community at large.

In a further embodiment, the target finding system includes at least four sensors disposed coplanar in a first plane and configured in a first Wheatstone bridge having a first sensing/zero output plane and a second Wheatstone bridge disposed in a second plane parallel to the first plane having a second sensing/zero output plane perpendicular to the first sensing/zero output plane. In this embodiment, the technique includes determining when a center axis of the electromagnetic signal is aligned with an intersection of a first zero-output plane and a second zero-output plane of the needle guide aperture by detecting when the center axis is in the first zero-output plane using the first Wheatstone bridge and detecting when the center axis is in the second zero-output plane using the second Wheatstone bridge. Such a system and technique provide a more accurate determination of a needle guide and surgical needle when performing a surgical procedure.

A target finding system identifies a surgical target such as a kidney stone by disposing an emitter such as a magnetic source behind or adjacent to the surgical target, and employing the circuit to identify an axis to the emitter, thus defining an axis or path to the surgical target. An array of sensors arranged in an equidistant, coplanar arrangement senses a signal indicative of a distance and direction to the emitter. In the case of a magneto resistor sensor, a variable resistance is responsive to the distance and direction from the magneto resistor to an emitter defined by a magnetic coil emitting a magnetic field. An equal signal from each of the coplanar sensors indicates positioning on an axis passing through a point central to the sensors and orthogonal to the plane.

In an example arrangement as discussed below, the sensor element is a magneto resistor sensor that varies resistance in response to a magnetic field. The magneto resistor sensor each define one leg of four legs in a Wheatstone bridge configuration. A Wheatstone bridge, as is known in the art, has four legs and is typically shown in a square or diamond arrangement. Electrically the Wheatstone bridge is connected to a voltage source such that the ratio of the resistance of two of the legs is equivalent to the ratio of resistance of the other two legs. In certain embodiments disclosed herein, the Wheatstone bridges physically have a true geometrically square (regular quadrilateral) spatial placement of the sensors. It is understood that other sensor arrangement are possible and that calibration methods known in the art can compensate for variations in sensor placement.

Configurations herein allow for determining a distance and direction from an axis passing through the middle of the square, hence being equidistant from each of the legs (assuming the sensor elements define similar positions around the square). In implementation, the resistive values of the magneto resistor are equal when aligned on a central axis normal to a plane defined by the magneto resistors (sensors). Hence an output signal defined by the difference of the voltage across each of the legs will be zero.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1A is a schematic diagram of a target finding system as disclosed herein;

FIG. 1B shows a context view of the target finding system of FIG. 1A;

FIG. 2 is a schematic diagram of the target finding system of FIG. 1 in operation; and

FIG. 3 shows a sensor array circuit of the target finding system in a bridge configuration;

FIG. 4A is a schematic diagram of an embodiment of alternate embodiment having orthogonal two bridge circuits;

FIG. 4B is a perspective view of the two orthogonal bridge circuits and two corresponding orthogonal sensing planes or the alternate embodiment of FIG. 4A;

FIG. 5 is a perspective view of two orthogonal sensing planes of the embodiment of FIG. 4B with a needle guide off axis;

FIG. 6 is a perspective view of two orthogonal sensing planes of the embodiment of FIG. 4B with an aligned needle guide; and

FIG. 7 is a flowchart of locating a surgical target using the target finding system of FIG. 1; and

FIG. 8 is a flowchart showing further details of locating a surgical target using the target finding system of FIG. 1;

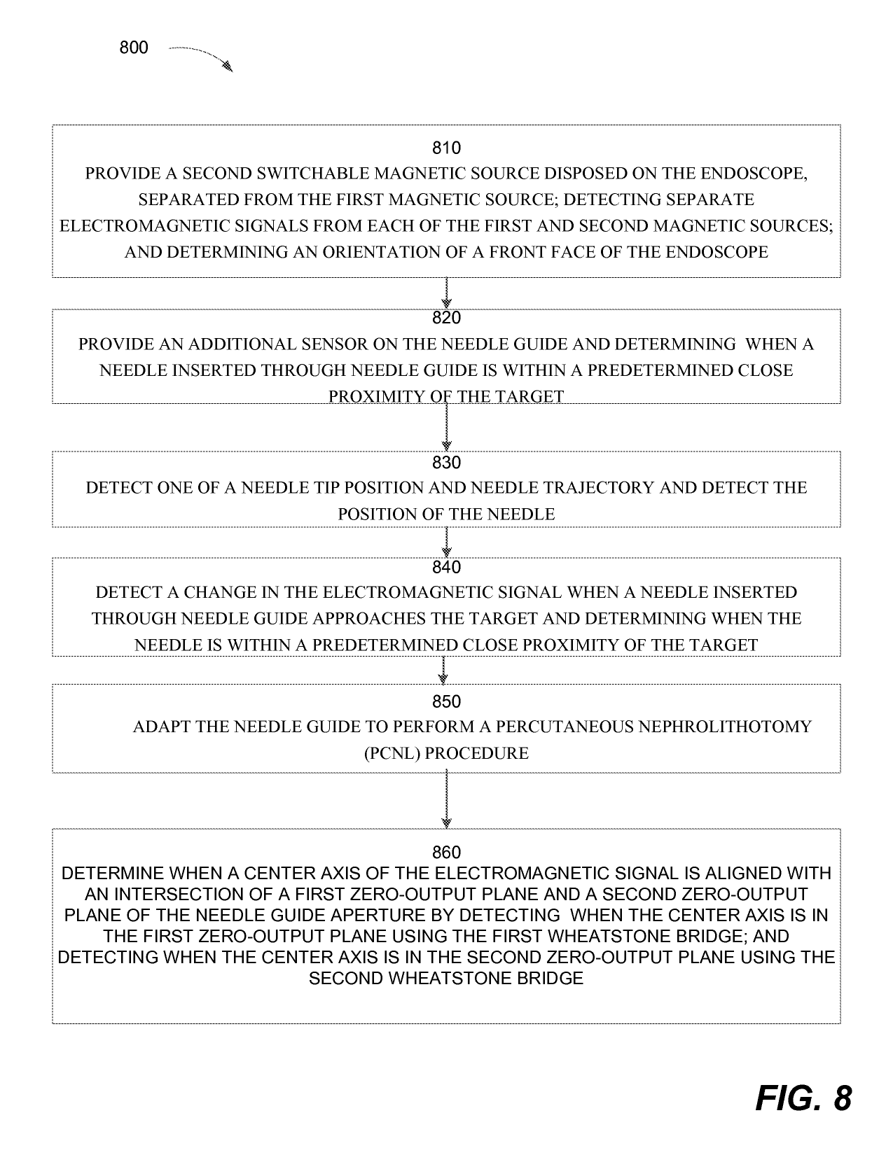

### DETAILED DESCRIPTION

The following examples and discussion illustrate various configurations of the disclosed approach. In the example configuration, the proposed approach facilitates the alignment of a percutaneous nephrolithotomy needle with the targeted calyx. Such alignment is a precise step during percutaneous nephrostomy, which prepares for a percutaneous nephrolithotomy (PCNL) procedure, more commonly referred to as a kidney stone removal. During such a procedure, the task of obtaining access manually requires substantial skill and can be particularly difficult when the collecting system is not dilated. Inaccurate placement of a surgical needle can create hazards, such as injuring renal blood vessels or adjacent organs, thus compromising the planned percutaneous procedure, as well as the clinical outcome of the patient.

In a general implementation, the target finding system for finding an internal surgical target such as the kidney stone, includes an Internal Target Object (ITO) including a signal source. The system further includes an External Target Finder (ETF) a detection system including a sensor circuit for localizing the coordinates of these in 3-D space around the patient, and a display system for the user. The ITO is an energy source that is detected and localized in 3-D space by the ETF detection system. In hand-held ETF embodiments, orientation and location information is sent to a user interface display system to provide guidance and feedback to an operator. In an automated robotic ETF embodiment, information is sent to controller to move and position the target finding system under a motion control system.

In embodiments directed to PCNL procedures, the inventors have observed that due the specifics of the procedure that a general positioning system is not required and that the electronics and controls can be simplified. For example the surgeon have access to one side of the surgical target (i.e., the kidney stone) using the ureteroscope. In these embodiments, simpler analog detection circuits and user interfaces can be used.

A controller and positioning system updates the user interface display system continuously, enabling the operator (e.g., a surgeon or user) to visualize the location and orientation of the vector relative to the point. This continuous stream of information provided to the operator by the system enables the needle to be directed and steered towards the internal target in real time. In other embodiments, the needle has two or more similar energy sources located along its length. Each of these sources are detected and localized in 3-D space by the detection system. Additionally the needle can include sensors. The spatial location information derived from for the multiple sources on the needle is provided to the controller and positioning system which generates a vector in 3-D space while the spatial location information for the internal target generates a point in 3-D space.

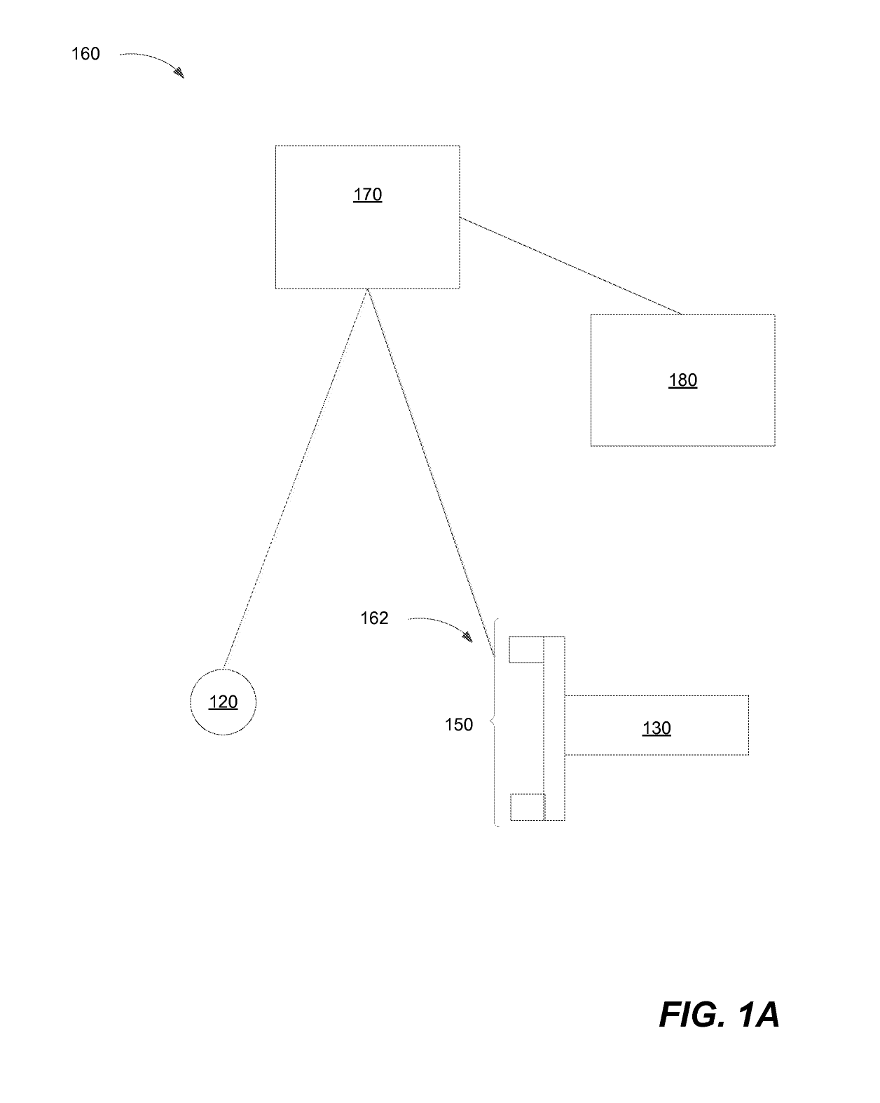

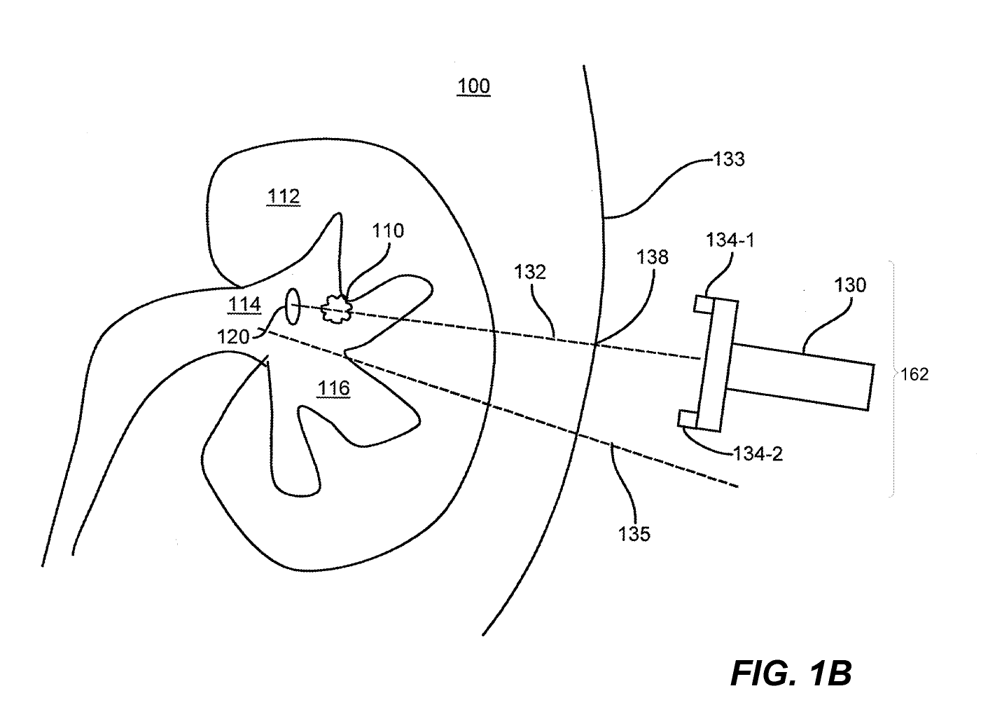

Now referring to FIG. 1A, an exemplary target finding system 160 includes two major sub-systems: an Internal Target Object (ITO) 120 (also referred to as signal source 120) and an External Target Finder (ETF) 162. The ETF 162 includes needle guide 130. The target finding system 160 further includes a controller and positioning system 170 (also referred to as a detector 170) a user interface (UI) 180 coupled to the detector 170. In one embodiment, the detector 170 and the user interface 180 are analog circuits. In an analog circuit embodiment, the UI 180 includes an array of LEDs or horizontal and vertical lines of LEDs providing a directional indication of how to move the ETF 162. In another embodiment, the detector 170 is a digital signal processor. In this embodiment, the UI 180 includes a three-dimension display directing the movement of the ETF 162.

The ITO 120 is a magnetic field generating device that is deployed through the working channel of a flexible ureteroscope, or as a built-in part inside the tip of the ureteroscope. At the tip of the ITO 120 is a small head that produces a magnetic field, static or pulsed. In one embodiment the ITO 120 includes a static magnetic field provided by a permanent magnet. In the permanent magnet embodiment no connection from the signal source 120 to the detector 170 is required. In another embodiment the ITO 120 includes a varying magnetic field provided by a current carrying coil which is connected to the detector 170.

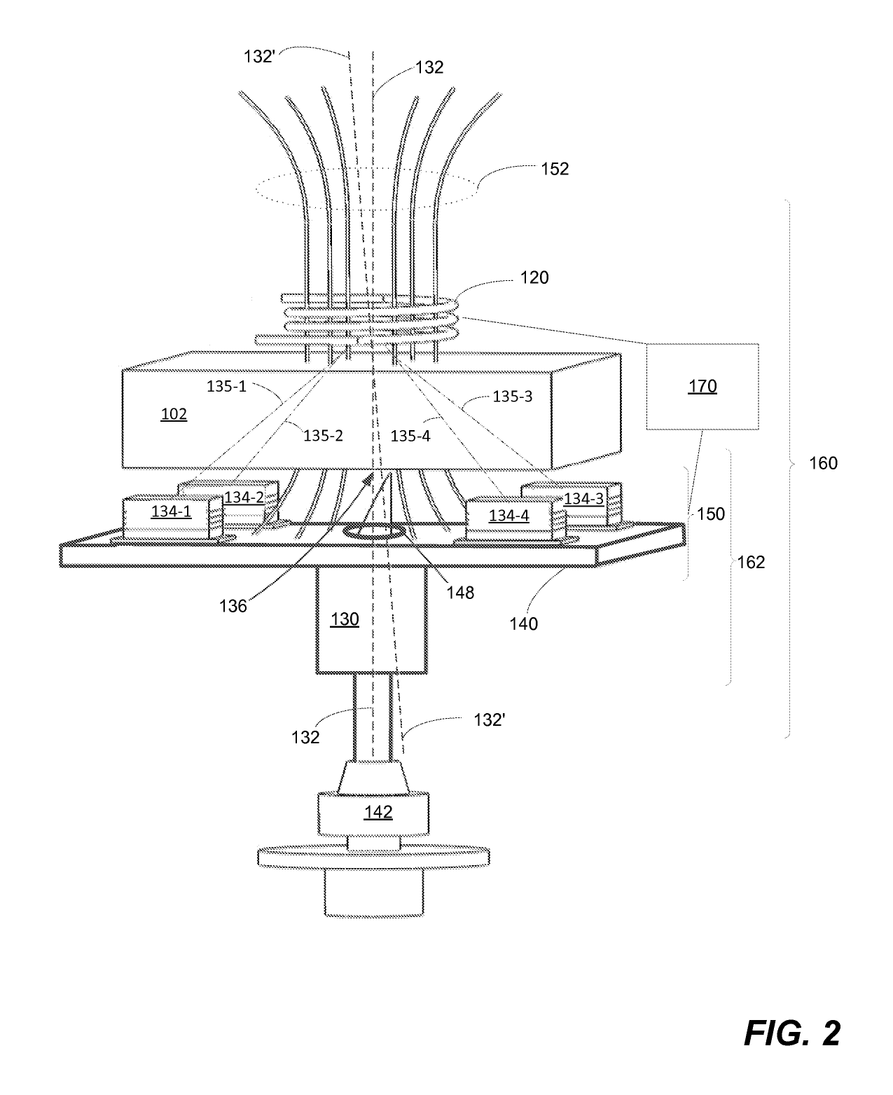

The ETF 162 may be a hand-held or robot/mechanical arm mounted magnetic field sensing device. In the one embodiment, the ETF 162 is instrumented such that when the center of the needle guide 130 is aligned with the magnetic field produced by the ITO 120, this configuration is detected by detector 170 and the user interface 180 signals that such alignment is established by feedback to the user of the target finding system 160. Feedback from the user interface 180 includes, but is not limited to, acoustical feedback (e.g. beeping, or changing tone), mechanical feedback (e.g. vibration), visual feedback (e.g. 2-D LED array or LCD display), or a combination thereof. In one embodiment, a guidance channel, located on the ETF 162, can guide an access needle 142 (FIG. 2) inserted in the needle guide 130 towards the calyx in the kidney inside the body.

The target finding system 160 operates based on the fact that human body is largely “transparent” to magnetic fields and the magnetic part of low frequency electromagnetic wave. Other forms of physical fields and energy propagation, such as radio frequency electromagnetic wave, IR radiation, ultrasonic wave, tend to be impeded by body tissues in various complex ways.

FIG. 1B shows a context view of a target finding system 160 including ITO 120 and ETF 162 of FIG. 1A. The ETF 162 includes at least four sensors 134-1-134-4 (134 generally). In one embodiment, the sensors 134 comprise magnetic sensors connected in a measuring bridge circuit. In another embodiment the sensors are solid state detectors configured in a multi-dimensional array. The sensors 134 are coupled to the detector 170.

In operation, under visual guidance through the ureteroscope, the tip of the ureteroscope is positioned in front of the stone infected calyx, and so is the tip of the ITO 120, positioned/oriented facing the surgical target 110, here a stone in the calyx. In one embodiment, when the ITO 120 is energized, it “paints” the target (e.g., the kidney stone). In another embodiment a small orientation sensor is built into a flexible ureteroscope to provide information from which the shape of the flexible ureteroscope inside body lumen can be determined. Under the direct observation through the endoscope, the surgeon or operator ascertains that ITO 120 is positioned behind the surgical target 110 (e.g., a kidney stone inside a calyx). Referencing the results from pre-operation radiological imaging of the affected kidney, the surgeon or operator will have a good idea on where to place the EFT 162 to search for the surgical target 110.

In the context of a kidney stone operation, a patient 100 undergoes a procedure for removal of a surgical target 110 such as a kidney stone from a kidney 112. The signal source 120 such as a magnetic coil or a permanent magnet is surgically disposed on a distal side 114 of the kidney stone 110. The signal source 120 may be disposed by any suitable method, typically by endoscopic instruments into a void 116 of the kidney 112. The ETF 162 is disposed externally on or near the surgical surface 133 (i.e. skin) of the patient 100. The ETF 162 locates an axis 132 to the signal source, by comparing distance and relative orientations from each of a plurality of sensors 134 to the signal source 120. It should be noted that the sensors 134 detect a magnetic field strength, which is related to the distance although based on the field orientation as well. Thus, the orientation of the sensors 134 seeks to locate the axis 132 by field strength indicative of orientation and distance from the signal source 120. By positioning the signal source 120 on the axis 132 and behind the kidney stone or other surgical target 110, the ETF 162 locates an insertion point 138 and angle on the axis 132 which can then be followed to reach the surgical target 110. The ETF 162 therefore ensures surgical insertion on the axis 132 for intersecting with the surgical target 110, improving upon conventional approaches which have the potential for inadvertent needle insertion along an incorrect path. Therefore, alignment on the axis 132 is facilitated by unbalanced sensor readings resulting from alignment along axes offset from 132, for example 135, as discussed further below. In one embodiment, the device of the present invention may work with a signal source 120 located at the distal tip of an endoscope. The endoscope allows for direct observation of the target and placement of the signal source directly behind the target stone 110.

One embodiment of the target finding system 160 is illustrated in FIG. 2. For clarity and compactness, the components are not shown in real dimensions and proportions. The ITO's 120 magnetic field generating component is isolated and represented by a coil, with all other parts on the ITO 120 and the ureteroscope are hidden. The various body tissues separating the ITO 120 and ETF162 are combined and represented as one barrier. FIG. 2 is a diagram of the target finding system 160 of FIG. 1 in operation. Referring to FIGS. 1 and 2, a generalized target finding system suitable for use with configurations herein is disclosed. In FIG. 2, the target finding system 160 (also referred to as the PCNL target finding (TF) system 160) employs at least four sensors 134-1 . . . 134-4, arranged in a square, arrangement on a planar base 140 (also referred to as a detector plate 140) of the ETF 162 to define a sensor circuit 150 defining an array of sensors 134 (sensor array circuit). The use of a planar base 140 and equidistant square arrangement provides for a uniform signal when the sensors are disposed orthogonally centered on the axis 132 passing through the center 148 of the planar base 140. In the example configuration, the signal source 120 is a magnetic coil and the sensors 134 are magneto-resistors that vary resistance in response to a magnetic field 152 emitted by the signal source 120. The distance from the sensors 134-1 . . . 134-4 is shown by dotted lines 135-1 . . . 135-4, respectively. When the distances 135-1 . . . 135-4 are equal, the response (e.g., resistance) of each of the sensors 134 is equal.

As disclosed herein, one embodiment includes a target finding system 160 tailored to a particular surgical procedure (PCNL) and the anatomy that is relevant to it. Such an implementation may not need extensive data processing and calculations and in one embodiment the ETF 162 is a self-contained hand-held analog electronic device. It is understood that the ETF 162 can be modified for other surgical procedures. Other embodiments include target finding/position locating/orientation determination functionalities that can be applied in more general situations (e.g., surgical procedures on different body organs for different treatment). These embodiments may require multiple channel data collection and processing efficiently through digital means.

In one embodiment, the ITO magnetic source 120 is a permanent magnet, if a permanent magnet is deemed safe and deliverable into the kidney. In another embodiment, the magnetic source 120 is a current carrying electric coil. The magnetic field could be static, or pulsed, or alternating. The field gradient could be also used in finding the relative distance and directionality between the source and sensors.

The target finding system 160 further includes the detector 170 coupled to the sensors 134 and a user interface (UI) 180 coupled to the detector 170. In operation, the detector 170 processes the signals from the sensors 134 and any other sensors located on the ETF 162 or the needle guide 130. The processed signal is sent to the UI 180 which provides audible and/or visual feedback.

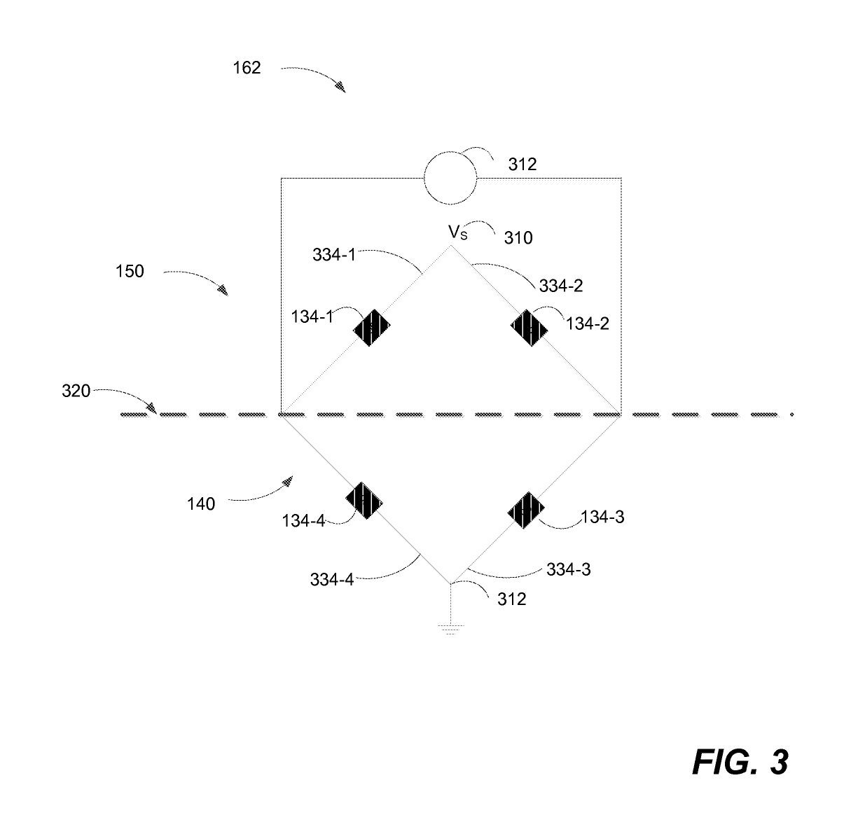

FIG. 3 shows a sensor array circuit 150 of the ETF 162. The sensors 134 are arranged in a bridge configuration. In the example arrangement, the sensors 134 are magneto-resistors which vary a resistance in response to magnetic field strength from the magnetic source 120. The sensor array circuit 150 includes four legs 334-1 . . . 334-4 (334 generally), each leg 334 having a sensor 134. The sensor array circuit 150 further includes a circuit 312 (also referred to as a signal conditioner) to measure any unbalanced signals in the bridge. Each leg 334 also has a voltage value V1 . . . V4 measured as the voltage drop across each leg 334. A source voltage Vₛ 310 provides a known voltage to each leg 334, and connects to the one side of the sensor array circuit 150 and the opposite side of the sensor array circuit 150, is connected to ground 312 for providing a voltage drop V1 . . . V4 value.

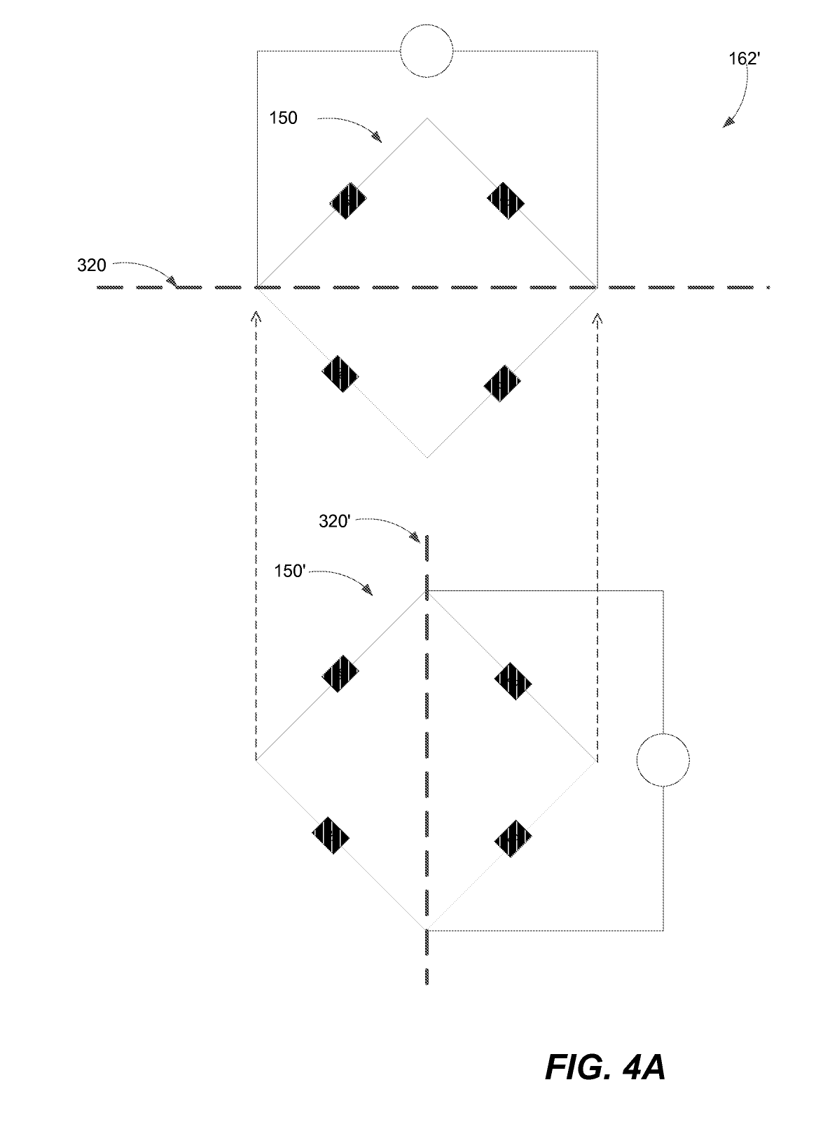

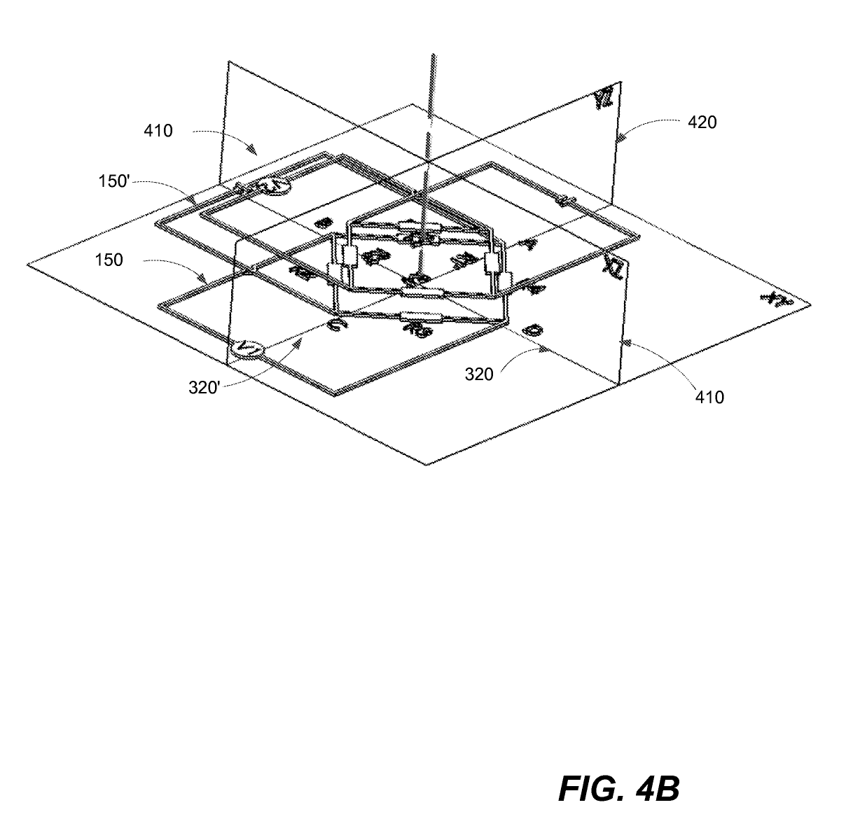

Line 320 represents the intersection of a sensing plane (shown and described below in conjunction with FIG. 4B) and the planar base 140. The sensing plane is orthogonal to the planar base 140. The target finding system 160 relies upon minute changes in the signal received from each of the sensors 134. Upon application of the source voltage Vs, a corresponding current I1 . . . I4 flows in each leg 334. The current in each leg 334 can be used to determine the corresponding field strength at the location of each sensor 134. Further details of embodiments of the sensor array circuit 350 can be found in earlier filed U.S. Provisional Patent Application having Ser. No. 61/783,607, filed Mar. 14, 2013, and entitled “SURGICAL POSITIONING CIRCUIT,” which is hereby incorporated by reference herein in its entirety.

Circuits constructed from resistor sensors configured in a bridge pattern are capable of aligning the plane these resistors are residing on with a physical field (the resistor sensor will respond to its field). In this circuit, all four resistors are live, sensors that change their resistance in response to the physical field they are subjected to, i.e., all four legs have common nominal resistance value, but that value varies according to local field strength. An axially symmetric gradient field, generated for example, by a magnetic field around a current-carrying coil, this circuit will help find the axial direction of the field. When all four sensors detect the same field strength, the field axis is perpendicular to the plane of the sensors and passes through the center of the square with a sensor at each corner. It is understood that a bridge of fixed and variable resistors can be used in place of each individual sensor in addition to other sensor configurations such as those described in Provisional Patent Application 61/783,607.

Now referring to FIG. 4A, an alternative ETF 162′ includes a pair of sensor array circuits 150 and 150′. In one embodiment, the sensors are, for example, Micro Magnetics, Inc. model STJ-240 magnetic sensors. The sensor array circuits 150 and 150′ can be co-located on a common plane or located on parallel planes. In this embodiment the sensor array circuits have corresponding sensing planes 410 and 420 (FIG. 4B) which are orthogonal. With a single sensor array circuit 150 the “axis on center” position and orientation is not the only “solution” when the bridge output is zero. For example, the output of the bridge in FIG. 3 will be zero, where the magnetic field axis produced by ITO 120 is anywhere inside (i.e., co-planar) a YZ sensing plane orthogonal to line 320.

Finding the field axis with a sensor array in a Wheatstone bridge configuration is enhanced by adding an additional Wheatstone bridge with sensor array circuit 150′. The Wheatstone bridge with sensor array circuit 150′ can co-exist on the same plane or parallel to with, and with their diagonals orthogonally placed, i.e. 90 degrees apart, as in shown in FIG. 4A. In this embodiment, the second bridge orthogonal to the first is added to better determine the location/orientation of the symmetric energy field. In another embodiment, one with sensor array circuit can be placed inside the other. Now referring to FIG. 4B, the alternative ETF 162′ including the pair of sensor array circuits 150 and 150′ (also referred to as bridges 150 and 150′) provide corresponding orthogonal sensing planes 410 and 420.

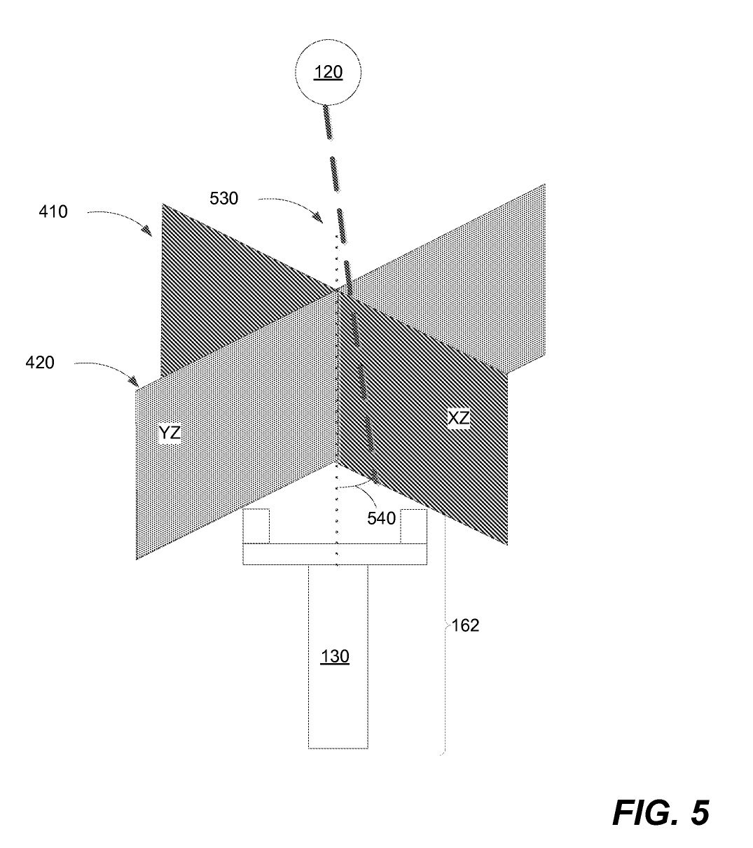

Now referring to FIG. 5, the two orthogonal sensing planes 410 and 420 (also referred to as zero output planes—where the corresponding bridge output is zero) of the embodiment of FIG. 4A are shown in a perspective view. Also shown is a needle guide 130, which here is off axis from the field axis 530 of the ITO 120 as indicated by angle 540. In one embodiment, the needle guide 130 is an articulable needle guide 130. The needle guide 130 itself, or the access needle 142 itself, will be instrumented such that its location and orientation will be monitored through orientation/gravity sensors mounted on the needle guide 130, or access needle 142. As the access needle 142 can be disposable, these sensors can reside on the articulable needle guide 130. In one embodiment, the ETF 162 detects when the needle is within close proximity of the target. This is accomplished using either an additional sensor or detection of a change in the electromagnetic signal when the access needle 142 approaches the target. In another embodiment, a ferromagnetic needle (not shown) is used and can be sensed by the ETF 162 or an additional sensor (not shown). It is understood that other techniques can be used to determine the directionality of the access needle 142 relative to the needle guide 130 in addition to relying on magnetic principles. For example, two rotational potentiometers in a gimbal arrangement and knowledge of the ETF 162 configuration can determine the relative position of the access needle 142.

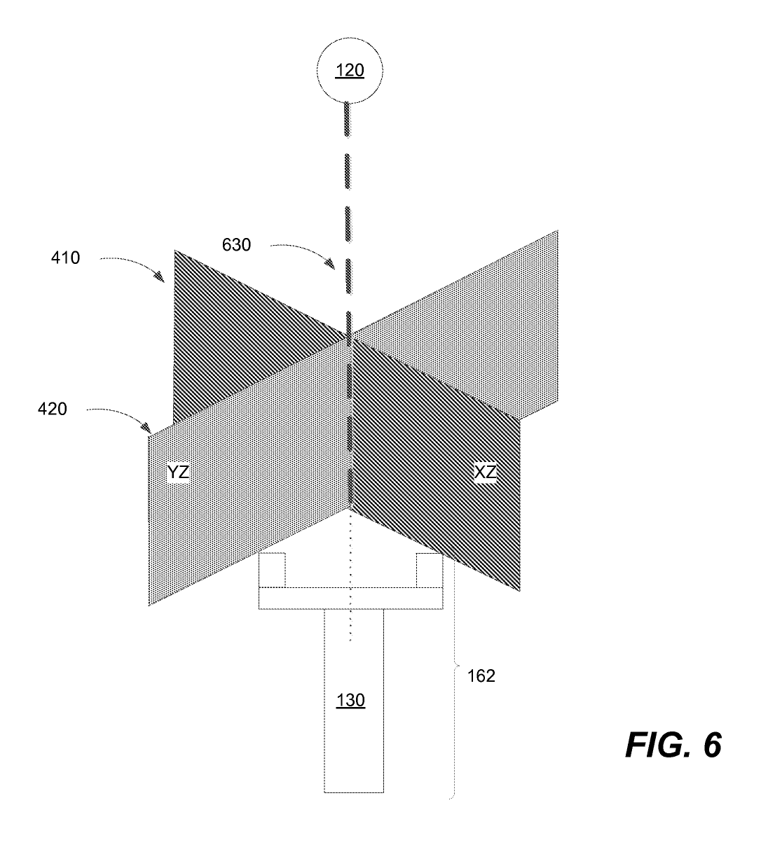

FIG. 6 is a diagram of two orthogonal sensing planes 410 and 420 of the embodiment of FIG. 4A with an indication of a field axis 630 of the axial symmetric magnetic field provided by ITO 120. . Here the needle guide 130 is aligned with the field axis 630 (i.e., when both Wheatstone bridge outputs 150 and 150′ are equal to zero). When bridge 150 yields an output of zero volts, the field axis must be in the YZ plane 520 When bridge 150′ yields zero volt output, the field axis must be in XZ plane 510. So when the outputs from both bridges are zero, the field axis 630 is in both YZ and XZ planes simultaneously, thus the field axis 630 is perpendicular to the sensor planes and goes through the center of the sensor arrays 150 and 150′ and therefore through the center of the needle guide 130. In a fixed needle guide embodiment (e.g., where the needle guide 130 is fixed relative to the bridges 150 and 150′) when aligned the field axis 630 should be co-linear with the intersection of the two zero-output planes XZ 410 and YZ 420, defined individually by the two orthogonal bridges 150 and 150′.

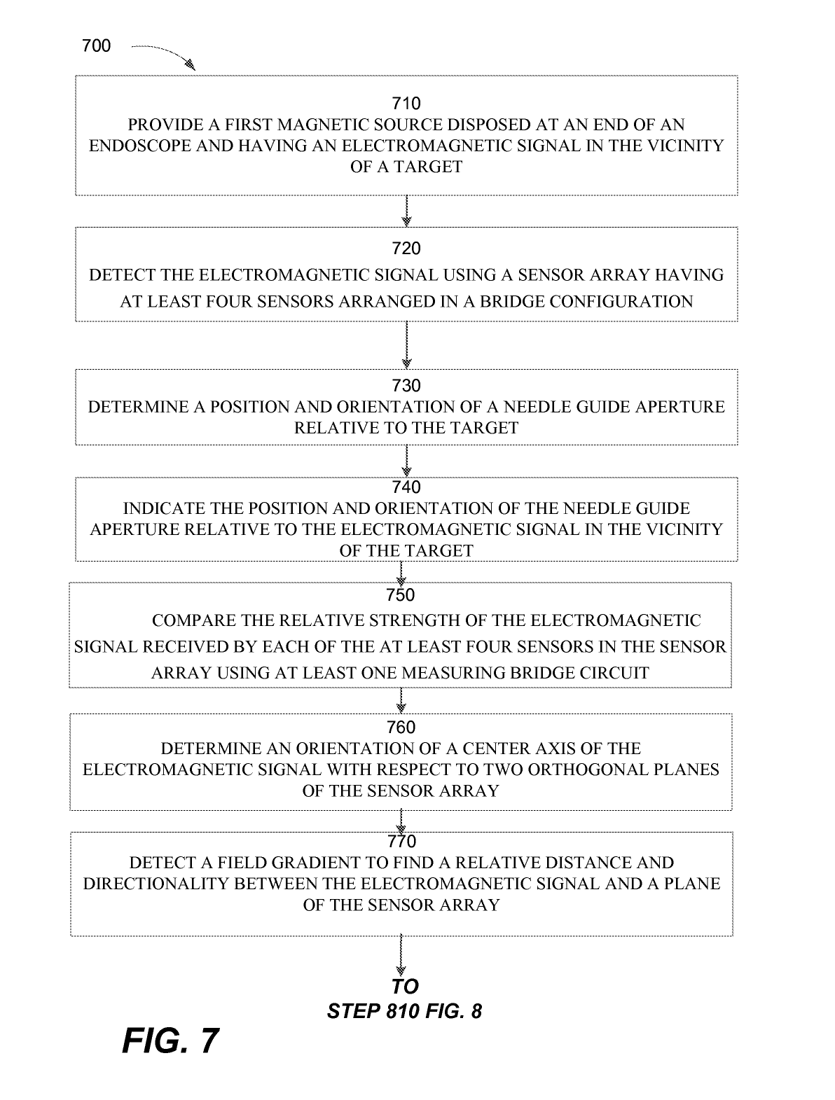

FIG. 7 shows a flowchart 700 of finding a surgical target using the target finding system 160 of FIG. 1. Referring to FIGS. 1 and 2, the method of finding a surgical target as disclosed and shown herein includes providing a first magnetic source disposed at an end of an endoscope and having an electromagnetic signal in the vicinity of a target as depicted at step 710. In one embodiment the signal source 120 is disposed on a distal side 114 of a surgical target 110.

As a surgeon or other operator orients the target finding system 160 on a proximate side of the surgical target 110, the electromagnetic signal is detected using a sensor array having at least four sensors arranged in a bridge configuration, as shown at step 720. At step 730, a position and orientation of a needle guide aperture relative to the target are determined, and at step 740, the position and orientation of the needle guide aperture relative to the electromagnetic signal in the vicinity of the target is indicated to the operator through the user interface 180.

At step 750, the relative strength of the electromagnetic signal received by each of the at least four sensors in the sensor array is compared using at least one measuring bridge circuit. An orientation of a center axis of the electromagnetic signal with respect to two orthogonal planes of the sensor array is determined at step 760. At step 770, a field gradient to find a relative distance and directionality between the electromagnetic signal and a plane of the sensor array is detected.

At step 810, a second switchable magnetic source is provided disposed on the endoscope, separated from the first magnetic source, separate electromagnetic signals from each of the first and second magnetic sources are detected and an orientation of a front face of the endoscope is determined. At step 820, an additional sensor is provided on the needle guide 130 and it is determined when the access needle 142 inserted through needle guide 130 is within a predetermined close proximity of the target. At step 830, a needle tip position or a needle trajectory is detected, and this facilitates detecting the position of the needle. In one embodiment, the depth of the access needle 142 is detected without the use of additional sensors. Here, the access needle 142 is advanced until the desired depth of the access needle 142 is indicated by the appearance of the liquid content inside the kidney at the proximal (external) opening of the needle by visual observation. In an embodiment using multiple sensors and signal sources the signal sources are operated at differing frequencies.

At step 840, a change in the electromagnetic signal is detected when the access needle 142 is inserted through needle guide 130 approaches the target and it is determined when the needle is within a predetermined close proximity of the target. Step 850 discloses adapting the needle guide 130 to perform a percutaneous nephrolithotomy (PCNL) procedure.

Using an embodiment similar to the system of FIG. 4A where the sensor array includes a first Wheatstone bridge in a first plane having a first zero-output plane, a second Wheatstone bridge disposed in a second plane parallel to the first plane having a second zero-output plane perpendicular to the first zero-output plane, it is determined when a center axis of the electromagnetic signal is aligned with an intersection of a first zero-output plane and a second zero-output plane of the needle guide aperture. This is accomplished by detecting when the center axis is in the first zero-output plane using the first Wheatstone bridge and detecting when the center axis is in the second zero-output plane using the second Wheatstone bridge at step 860.

The disclosed target finding system for detection and spatial location of any of the targets could be achieved through a variety of physical detection mediums. Any of the aforementioned sources could be a single point emitter. This generally employs electrical or acoustic energy source which could be but is not limited to magnetic, ultrasound, high frequency RF energy, low frequency RF energy, time varying magnetic fields, or time varying electrical fields. The target finding system could comprise a plurality of appropriate, non co-located energy sensors located outside the body. Each sensor would utilize some detection scheme to determine the linear distance between it and the emitting energy source. This could be accomplished by detection using an appropriate energy receiver and multiple means including but not limited to: 1) utilizing phase information of the signal driving the source as the phase varies with the time of flight, and 2) measuring the change in the amplitude of the received energy as it varies with distance between the emitter and receiver, or 3) utilizing the trip time of flight of a change in signal amplitude such as a chirp or pulse. Other measuring schemes for determining the distance between the sensor and emitter based on advance phased-array detection may also be envisioned.

In operation, distance information from each of the plurality of sensors 134 in the detector system is used to localize a given signal source 120 in 3-D space. In the example approach this is accomplished by arranging the detectors into an array with known spatial locations and employing triangulation with the measured distances. More advanced methods are possible, for example using phased-array schemes such as phased array antenna or synthetic aperture antenna methods. In the example configuration of the PCNL Target Finding system (PCNL TF) includes two major subsystems, an Internal Target Object (ITO) (defined by the signal source 120) and an External Target Finder (ETF) 162, defined by the target finding system 160. The ITO 120 is a magnetic field generating device that is deployed through the working channel of a flexible ureteroscope, or as a built-in part inside the tip of the ureterosocope. At the tip of the ITO 120 is a small head that produces a magnetic field, static or pulsed. Under visual guidance through the ureteroscope, the tip of the ureteroscope is positioned in front of the stone infected calyx, and so is the tip of the ITO 120, positioned/oriented facing the stone in the calyx. The ETF 162 is a hand-held or robot/mechanical arm mounted magnetic field sensing device. The ETF 162 is instrumented such that when the center of the ETF 162 is aligned with the magnetic field produced by the ITO 120, the ETF 162 will signal such alignment is established. A center guidance channel then can guide the access needle towards the calyx in the kidney inside the body. The entire system works based on the fact that human body is largely “transparent” to static magnetic fields and the magnetic part of low frequency electromagnetic wave, in contrast with conventional x-ray or ultrasound approaches which encounter difficulty in distinguishing soft tissue structures. In this configuration, the ITO's 120 magnetic field generating component is isolated and represented by a coil defining the signal source 120, with all other parts on the ITO 120 and the ureterosocope omitted for clarity. The various body tissues separating the ITO 120 and ETF 162 is combined and represented as one barrier as surgical surface 133, now described in operation in greater detail.

In an alternate arrangement, the external target finding system could be mechanically coupled to simplify the system overall. In this case the PCNL needle would be coupled through mechanical means directly or indirectly to the detection system. This eliminates some of the information necessary in determining the relative spatial locations of the internal target and the external targets defining the position of the PCNL needle. The location and orientation of the needle relative to the detection system would be predetermined. This information would then be used to simplify the solution of the equations involved in triangulation between the internal target and the external targets. This could be accomplished through a rigid mounting mechanism that would align the needle in a fixed orientation relative to the sensors in the detection system. The PCNL needle and detection assembly as a combined unit could then be steered in real time towards the internal target. In a less restricted implementation the mechanical coupling would allow one or more degrees of freedom between the PCNL needle and the detection system. This could be accomplished with an articulation mechanism and appropriate indexing sensors or using stepper motors or other similar methods. This information giving the relative location and orientation of the PCNL needle relative to the sensors in the detection system would then become part of the solution of the triangulation equation to locate these relative to the internal target.

In other embodiments, orientation information from orientation/gravity sensor can be combined with position/orientation information from magnetic sensing of the target finding system to provide shape visualization system for flexible endoscopes.

Configurations disclosed herein include at least some features that may be implemented by a computer or similar processor based set of programmed instructions. Alternate configurations of the invention may therefore include a multiprogramming or multiprocessing computerized device such as a multiprocessor, controller or dedicated computing device in either a handheld, mobile, or desktop form or the like configured with software and/or circuitry (e.g., a processor as summarized above) to process any or all of the method operations disclosed herein as embodiments of the invention. Still other embodiments of the invention include software programs such as a Java Virtual Machine and/or an operating system that can operate alone or in conjunction with each other with a multiprocessing computerized device to perform the method embodiment steps and operations summarized above and disclosed in detail below. One such embodiment comprises a computer program product that has a non-transitory computer-readable storage medium including computer program logic encoded as instructions thereon that, when performed in a multiprocessing computerized device having a coupling of a memory and a processor, programs the processor to perform the operations disclosed herein as embodiments of the invention to carry out data access requests. Such arrangements of the invention are typically provided as software, code and/or other data (e.g., data structures) arranged or encoded on a computer readable medium such as an optical medium (e.g., CD-ROM), floppy or hard disk or other medium such as firmware or microcode in one or more ROM, RAM or PROM chips, field programmable gate arrays (FPGAs) or as an Application Specific Integrated Circuit (ASIC). The software or firmware or other such configurations can be installed onto the computerized device (e.g., during operating system execution or during environment installation) to cause the computerized device to perform the techniques explained herein as embodiments of the invention.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A target finding system, comprising:
an internal target object (ITO) disposed at an end of an endoscope providing an electromagnetic signal;
an external target finder (ETF) comprising:
a needle guide aperture; and
at least four sensors arranged symmetrically around the needle guide aperture, the sensors configured to receive the electromagnetic signal from the ITO;
a detector coupled to the at least four sensors to determine an orientation and a position of a needle guide aperture relative to the ITO; and
a user interface connected to the ETF to indicate the orientation and the position of the needle guide aperture relative to the ITO.

2. The system of claim 1, wherein each of the at least four sensors comprise a magnetic sensor connected in at least one measuring bridge circuit and the detector and the user interface comprises an analog circuit.

3. The system of claim 1, wherein the ITO comprises a first magnetic source.

4. The system of claim 3, wherein the first magnetic source comprises one of:
a static magnetic field provided by a permanent magnet; and
a varying magnetic field provided by a current carrying coil.

5. The system of claim 4, wherein the endoscope further comprises:
a second magnetic source; and
wherein one of the first magnetic source and the second magnetic source is a switchable magnetic source.

6. The system of claim 4, further comprising a digital signal processor coupled to the at least four sensors and the user interface.

7. The system of claim 1, wherein the endoscope is a ureteroscope.

8. The system of claim 1, wherein each of the at least four sensors comprises a plurality of solid state detectors configured in a multi-dimensional array.

9. The system of claim 1, wherein the needle guide comprises at least one sensor disposed thereon to provide a location and an orientation of a needle disposed within the needle guide.

10. The system of claim 1, wherein the at least four sensors are disposed coplanar in a first plane and configured in a first Wheatstone bridge having a first sensing plane; and
further comprising a second Wheatstone bridge disposed in a second plane parallel to the first plane having a second sensing plane perpendicular to the first sensing plane.

11. A method of finding a target comprising:
providing a first magnetic source disposed at an end of an endoscope and having an electromagnetic signal in a vicinity of a target;
detecting the electromagnetic signal using a sensor array having at least four sensors arranged in a bridge configuration;
determining a position and orientation of a needle guide aperture relative to the target; and
indicating the position and orientation of the needle guide aperture relative to the electromagnetic signal in the vicinity of the target.

12. The method of claim 11 further comprising comparing a relative strength of the electromagnetic signal received by each of the at least four sensors in the sensor array using at least one measuring bridge circuit.

13. The method of claim 12 further comprising determining an orientation of a center axis of the electromagnetic signal with respect to two orthogonal planes of the sensor array.

14. The method of claim 12 further comprising detecting a field gradient to find a relative distance and directionality between the electromagnetic signal and a plane of the sensor array.

15. The method of claim 11 further comprising:
providing a second switchable magnetic source disposed on the endoscope, separated from the first magnetic source;
detecting separate electromagnetic signals from each of the first and second magnetic sources; and
determining an orientation of a front face of the endoscope.

16. The method of claim 11 further comprising:
providing an additional sensor on the needle guide; and
determining when a needle inserted through needle guide is within a predetermined close proximity of the target.

17. The method of claim 16 wherein in the needle guide aperture is movable within a detector plate;
detecting one of:
a needle tip position; and
a needle trajectory; and
detecting the position of the needle.

18. The method of claim 11 further comprising:
detecting a change in the electromagnetic signal when a needle inserted through needle guide approaches the target; and
determining when the needle is within a predetermined close proximity of the target.

19. The method of claim 11 further comprising adapting the needle guide to perform a percutaneous nephrolithotomy (PCNL) procedure.

20. The method of claim 11 wherein the sensor array comprises:
a first Wheatstone bridge in a first plane having a first zero-output plane;
a second Wheatstone bridge disposed in a second plane parallel to the first plane having a second zero-output plane perpendicular to the first zero-output plane;
the method further comprising determining when a center axis of the electromagnetic signal is aligned with an intersection of a first zero-output plane and a second zero-output plane of the needle guide aperture by:
detecting when the center axis is in the first zero-output plane using the first Wheatstone bridge; and
detecting when the center axis is in the second zero-output plane using the second Wheatstone bridge.
